(19) United States
(12) Patent Application Publication
(10) Pub. No.: US 2004/0121030 A1
(43) Pub. Date: Jun. 24, 2004

(54) Extract from the pods of lupin seeds containing lupeol

(76) Inventors: Antoine Piccirilli, Versailles (FR); Jacques Legrand, Neuilly Sur Eure (FR); Nicole Broutin, Alluyes (FR)

Correspondence Address: FOLEY AND LARDNER, SUITE 500, 3000 K STREET NW, WASHINGTON, DC, 20007, US

(21) Appl. No.: 10/473,861

(22) PCT Filed: Apr. 2, 2002
(86) PCT No.: PCT/FR02/01130

(30) Foreign Application Priority Data
Apr. 3, 2001 (FR) 01/04493

(51) Int. Cl. A61K 35/78; C07C 35/21

(52) U.S. Cl. 424/757

(57) ABSTRACT

The invention relates to an extract from the pods of lupin seeds containing lupeol, more particularly an extract wherein the content thereof by weight is greater than 30%, preferably greater than 50%, even more advantageously 70-100% The invention also relates to a method for obtaining said extract.

The present invention relates to an extract from the pods of lupin seeds containing lupeol, in particular a lupeol-rich extract. The invention also relates to a method for obtaining such an extract.

Lupin is quite a widespread plant, which is found in Europe, in Asia, and in North and South America. This plant is a close relative of the pea, of the broad bean, of soybean and of the French bean. Several species of lupin can be mentioned as being the most well-known: lupinus albus (white lupin), lupinus angustifolius (blue lupine), lupinus luteus (yellow lupin), lupinus mutabilis (pearl lupin), lupinus graecus, lupinus micranthus Guss, lupinus hispanicus, lupinus pilosus, lupinus cosentinii, lupinus atlanticus, lupinus princei and lupinus somaliensis. One of the most common species in Europe is sweet white lupin (lupinus albus), in particular the variety Ares which exhibits the pauper gene.

Although lupin seeds are conventionally used as fertilizer and also in human and animal foodstuffs for their high protein content, the pods or skins of lupin seeds have only been very rarely used in industry. They nevertheless constitute a natural source rich in lupeol.

Lupeol (1) belongs to the triterpene family, and more particularly to that of the triterpenic alcohols.

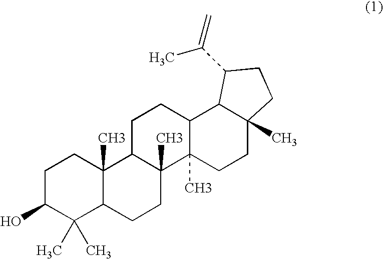

Lupeol is of definite value by virtue of its many biological activities. It is in particular known for its anti-inflammatory (Singh S. et al., Filoterapia, 1997, 68, No. 1, 9) and analgesic (De Miranda A. L. et al., Planta Med, 2000, 66(3), 284) properties, its nephroprotective action with respect to heavy metals (Nagaraj M. et al., J. Appl. Toxicol, 2000, 20(5), 413), its antihistamine action (De Medrano Villar M. J. et al., Methods Find Exp. Clin. Pharmacol., 1997, 19, No. 8, 515), and its antimitotic (Zachariah R. et al., Indian J. Pharm. Sci., 1994, 56, No. 4, 129) and antiviral (Kahlos K., Filoterapia, 1996, 67, No. 4, 344) activities.

Lupeol can also be used as a synthesis intermediate, in particular for preparing phytohormones and steroid analogs.

Lupeol is present in many plants, such as Aloe vera or the bark of Crataeva nurvala. It has been isolated on several occasions, from various plants such as “Bresk”. However, it has never been extracted from lupin pods. A lupin oil containing lupeol was extracted from lupin seeds in patent FR 2 762 512, but the oil then came from seeds which had been peeled beforehand and which had therefore had their pods removed, and the lupeol contained in the oil showed a maximum content of 0.5% by weight relative to the total composition of the oil.

Now, since lupin pods constitute a potential source rich in lupeol, which is cheap and much more readily available than most of its homologs, there was thus a need to isolate an extract from the pods of lupin seeds, in particular an extract rich in lupeol, and to develop a method for obtaining such an extract. Lupin in general, and white lupin (lupinus albus) in particular, is in fact an oil and protein yielding plant grown on a large scale, with modern agricultural techniques, unlike less widespread plants which contain lupeol, like Aloe vera and Crataeva nurvala.

A subject of the present invention is thus an extract from the pods of lupin seeds containing lupeol. Advantageously, the extract according to the present invention has a lupeol content of greater than 30% by weight, preferably greater than 50% by weight. Even more advantageously, the extract from the pods of lupin seeds according to the present invention has a lupeol content of between 70 and 100%.

For the purpose of the present invention, the term “pod of lupin seed” is intended to mean the skin surrounding the seed. The pods represent on average 15% by weight of the dry seed.

The extract according to the present invention is prepared from pods of lupin seeds derived from mechanical peeling of the seeds.

In a particular embodiment of the present invention, the lupin is chosen from the group consisting of lupinus angustifolius, lupinus albus, lupinus luteus, lupinus mutabilis, lupinus graecus, lupinus micranthus Guss, lupinus hispanicus, lupinus pilosus, lupinus cosentinii, lupinus atlanticus, lupinus princei and lupinus somaliensis. According to the present invention, the lupin is advantageously lupinus albus (sweet white lupin), of the European type, preferably the variety Ares carrying the pauper gene.

A subject of the present invention is also a method for obtaining an extract from the pods of lupin seeds according to the present invention.

Advantageously, the method according to the present invention comprises at least the series of following steps:

grinding the lupin pods,

extracting the total lipids contained in the lupin pods ground using an organic solvent chosen from the group consisting of aliphatic alkanes, aromatic alkanes, aliphatic alcohols, and halo-derivatives thereof, and

purifying the lipids obtained so as to obtain a lupeol-rich extract.

The pods isolated from the seeds are ground using a cylinder mill or a hammer mill. The method for obtaining a lupeol-rich extract according to the present invention is based on the extraction of the lipids contained in the lupin pods using an organic solvent. Advantageously, the organic solvent is hexane. The purified pods generally have a total lipid content of between 0.5 and 5% by weight relative to the total weight of the pods.

For the purpose of the present invention, the term “lupeol-rich” extract is intended to mean an extract having a lupeol content of greater than 30% by weight, advantageously greater than 50% by weight, and even more advantageously of between 70 and 100% by weight.

The purification of the lipids obtained in order to obtain a lupeol-rich extract according to the present invention is alternatively carried out by methods A, B and C described below.

In a particular embodiment of the present invention (method A), the purification of the total lipids extracted from the lupin pods comprises at least the series of following steps:

concentration of the reaction medium containing the lipids by evaporation of the organic solvent under vacuum in order to obtain a lipid content of between 0.1 and 90% by weight, advantageously between 10 and 30% by weight, even more advantageously 20% by weight, and

crystallization.

According to the present invention, it is thus possible to directly obtain a lupeol-rich extract by direct crystallization from the organic solution obtained during the step for extracting the total lipids present in the lupin pods. After the reaction medium containing the lipids has been concentrated, the solution is then cooled in order to initiate the crystallization of the lupeol. The lupeol-rich extract obtained can then be purified by one or more step(s) of recrystallization, in particular from hexane.

In another embodiment of the present invention (method B), the purification of the total lipids extracted from the lupin pods comprises at least the series of following steps:

evaporation of the solvent under vacuum, and then dissolution in an aliphatic alcohol,

saponification,

crystallization by cooling, and

filtration and/or washing and/or spin-filter drying.

According to the present invention, the aliphatic alcohol is advantageously ethanol and the solvent used in the saponification step is advantageously alcoholic potassium hydroxide. After saponification, the aqueous-alcoholic solution obtained is subjected to fractionated crystallization by cooling. After filtration and/or washing and/or spin-filter drying, the solid extract obtained can have a lupeol content of at least 30% by weight, advantageously of at least 50% by weight, even more advantageously of at least 60% by weight. The lupeol-rich extract obtained can then be purified by one or more step(s) of recrystallization, in particular from hexane.

In another embodiment of the present invention (method C), the purification of the total lipids extracted from the lupin pods comprises at least the series of following steps:

evaporation of the solvent under vacuum, then dissolution in an aliphatic alcohol,

saponification,

liquid-liquid extraction of the unsaponifiable material in the lupin pod lipids using an organic solvent chosen from the group consisting of aliphatic alkanes and halo-derivatives thereof, and

purification of the unsaponifiable material by washing, preferably with water, and then evaporation of the solvents under vacuum and drying.

According to the present invention, it is thus possible to obtain a lupeol-rich extract by saponifying the reaction medium containing the total lipids extracted from the lupin pods, and then extracting the unsaponifiable fraction of the lupin pod lipids.

For the purpose of the present invention, the term “unsaponifiable” is intended to mean the fraction of a fatty substance which, after prolonged action of an alkaline base, remains insoluble in water and can be extracted with an organic solvent.

According to the present invention, the liquid-liquid extraction is carried out in a pulsed counterflow column in the presence of a solvent having great affinity with respect to the unsaponifiable material, such as aliphatic alkanes and halo-derivatives thereof. Advantageously, according to the present invention, the organic solvent for the liquid-liquid extraction step is dichloroethane. The organic phase at the column outlet is then washed with water and then evaporated under vacuum, and the residue is dried. The lupeol-rich extract obtained can then be purified by one or more step(s) of recrystallization, in particular from hexane.

All the steps of methods according to the present invention are well known to those skilled in the art.

The following examples are given without limitation and illustrate the present invention.

### EXAMPLES OF THE IMPLEMENTATION OF THE INVENTION

The raw material used consists of the pods of seeds from sweet white lupin of the variety Ares, carrying the pauper gene. The total lipid content of the lupin pods is equal to 1.8% by weight.

1. Extraction of Total Lipids:

75 kg of lupin pods are pre-ground using a hammer mill. The ground material obtained is then loaded into a GUEDU agitated filter in order to undergo 8 successive washes with hexane (90 liters of hexane used per wash). The extraction temperature is fixed at 45° C. Each wash comprises: fresh solvent being introduced into the filter, agitation of the suspension for 15 minutes and, finally, filtration for 15 minutes in order to recover the miscella. At the end of the process, the delipidized lupin pods are steam-blasted, under vacuum, at a temperature of 50° C., in order to extract the residual miscella from the cake. The miscella phases are finally pooled, and then evaporated under vacuum, by injection of steam under vacuum, at 120° C. This operation is controlled so as to obtain a hexane-based solution A having a total lipid content of between 15 and 30%, preferably 20%, by weight.

2. Lupeol Extraction:

2.1 Method A:

The hexane-based solution A, obtained during the total lipid extraction, is cooled to 20° C. and left to stir slowly for 12 hours. It is then filtered through a büchner funnel so as to recover the lupeol-rich crystallized extract. This extract is finally washed with cold hexane (15° C.) and then dried at 60° C. in a vacuum oven for 48 hours.

The extract A obtained is in the form of a light yellow powder having a 76% lupeol content. The overall lupeol extraction yield is 72%.

2.2 Method A Followed by a Recrystallization Step:

500 g of the extract A obtained in Example 1 are purified by recrystallization from hexane. The solid extract is in fact dissolved in hexane (solution at 10% by weight). The mixture is brought to 70° C., at reflux, for 15 minutes, until the extract is completely solubilized. The temperature is then returned to 20° C. and maintained with slow stirring until complete precipitation of the lupeol. The latter is then filtered off, washed with cold hexane, and then finally dried at 60° C. in a vacuum oven for 48 hours.

The extract B obtained is in the form of a white powder having a 95% lupeol content. The overall lupeol extraction yield is 65%.

2.3 Method B:

2 kg of hexane-based solution A obtained during the total lipid extraction are completely evaporated, under a vacuum of 200 mbar and by injection of live steam at 120° C. The solid obtained is then taken up with 900 ml of ethanol. This new ethanolic solution is then saponified in a stirred, jacketed reactor in the presence of 240 g of 50% alcoholic potassium hydroxide. The saponification reaction is carried out at reflux for 4 hours and at a temperature of 70° C. The soapy solution is then returned to a temperature of 20° C. and maintained with slow stirring for 24 hours, until complete precipitation of the lupeol. The latter is then filtered, washed with cold hexane, and then finally dried at 60° C. in a vacuum oven for 48 hours.

The solid extract obtained is in the form of a whitish powder having an 84% lupeol content. The overall lupeol extraction yield is 91%.

2.4 Method C:

2 kg of hexane-based solution A obtained during the total lipid extraction are completely evaporated, under a vacuum of 200 mbar, by injection of live steam at 120° C. The solid obtained is then taken up with 900 ml of ethanol. This new ethanolic solution is then saponified in a stirred, jacketed reactor, in the presence of 240 g of 50% alcoholic potassium hydroxide. The saponification reaction is carried out at ref lux for 4 hours and at a temperature of 70° C. The soapy solution obtained is then diluted with softened water (50/50 dilution by volume). This new aqueous-alcoholic solution is then sent to the bottom of a pulsed counterflow column. As regards the top of the column, it is fed with 1,2-dichloroethane used as lupeol extraction solvent. The organic phase obtained then undergoes the following conventional single purification operations:

1) washing with water in a counterflow column

2) evaporation under vacuum in a rotary evaporator

3) drying of the residue in a vacuum oven (70° C., 48 hours).

The extract C obtained is in the form of a light yellow powder having an 85% lupeol content. The overall lupeol extraction yield is 87%.

2.5 Method C Followed by a Recrystallization Step:

200 g of extract C obtained in Example 3 can be purified by recrystallization from hexane. The solid extract is in fact dissolved in hexane (solution at 10%). The mixture is brought to 70° C. at reflux for 15 minutes, until complete solubilization of the extract. The temperature is then returned to 20° C. and maintained with slow stirring until complete precipitation of the lupeol. The latter is then filtered, washed with cold hexane, and then finally dried at 60° C. in a vacuum oven for 48 hours.

The extract D obtained is in the form of a white powder having a 96.5% lupeol content. The overall lupeol extraction yield is 70%.

1. An extract from the pods of lupin seeds containing lupeol.

2. The extract as claimed in claim 1, characterized in that it has a lupeol content of greater than 30% by weight, preferably greater than 50% by weight.

3. The extract as claimed in either one of claims 1 and 2, characterized in that it has a lupeol content of between 70 and 100% by weight.

4. The extract as claimed in any one of claims 1 to 3, characterized in that the lupin is chosen from the group consisting of lupinus angustifolius, lupinus albus, lupinus luteus, lupinus mutabilis, lupinus graecus, lupinus micranthus Guss, lupinus hispanicus, lupinus pilosus, lupinus cosentinii, lupinus atlanticus, lupinus princei and lupinus somaliensis.

5. The extract as claimed in claim 4, characterized in that the lupin is lupinus albus, preferably the variety Ares carrying the pauper gene.

6. A method for obtaining an extract as claimed in any one of the preceding claims.

7. The method as claimed in claim 6, characterized in that it comprises at least the series of following steps:
grinding the lupin pods,
extracting the total lipids contained in the lupin pods ground using an organic solvent chosen from the group consisting of aliphatic alkanes, aromatic alkanes, aliphatic alcohols, and halo-derivatives thereof, and
purifying the lipids obtained so as to obtain a lupeol-rich extract.

8. The method as claimed in claim 7, characterized in that the organic solvent is hexane.

9. The method as claimed in either one of claims 7 and 8, characterized in that the purification of the lipids obtained comprises at least the series of following steps:
concentration of the reaction medium containing the lipids by evaporation of the organic solvent under vacuum in order to obtain a lipid content of between 0.1 and 90% by weight, advantageously between 10 and 30% by weight, even more advantageously 20% by weight, and
crystallization.

10. The method as claimed in either one of claims 7 and 8, characterized in that the purification of the lipids obtained comprises at least the series of following steps:
evaporation of the solvent under vacuum, and then dissolution in aliphatic alcohol,
saponification,
crystallization by cooling, and
filtration and/or washing and/or spin-filter drying.

11. The method as claimed in either one of claims 7 and 8, characterized in that the purification of the lipids obtained comprises at least the series of following steps:
evaporation of the solvent under vacuum, then dissolution in an aliphatic alcohol,
saponification,
liquid-liquid extraction of the unsaponifiable material in the lupin pods using an organic solvent chosen from the group consisting of aliphatic alkanes and halo-derivatives thereof, and
purification of the unsaponifiable material by washing, and then evaporation of the solvents under vacuum and drying.

12. The method as claimed in claim 11, characterized in that the organic solvent for the liquid-liquid extraction step is dichloroethane.

13. The method as claimed in any one of claims 9 to 12, characterized in that it also comprises one or more final recrystallization step(s).
